(19) United States
(12) Patent Application Publication
(10) Pub. No.: US 2024/0122495 A1
(43) Pub. Date: Apr. 18, 2024

(54) Detecting a Specified Substance in an Examination Object

(71) Applicant: Siemens Healthcare GmbH, Erlangen (DE)

(72) Inventors: Daniel Nicolas Splitthoff, Uttenreuth (DE); Heiko Meyer, Uttenreuth (DE); Thomas Vahle, Nürnberg (DE); Florian Maier, Buckenhof (DE); Wei Liu, Erlangen (DE); Christianne Leidecker, Nürnberg (DE); Gregor Michael Körzdörfer, New York, NY (US); Peter Gall, Uttenreuth (DE); Daniel Polak, Erlangen (DE)

(73) Assignee: Siemens Healthcare GmbH, Erlangen (DE)

(21) Appl. No.: 18/379,438

(22) Filed: Oct. 12, 2023

(30) Foreign Application Priority Data
Oct. 13, 2022 (EP) 22201229.6

(51) Int. Cl. A61B 5/055; A61B 5/00

(52) U.S. Cl. CPC A61B 5/055; A61B 5/7267; A61B 5/7285

(57) ABSTRACT

Techniques are described for detecting a specified substance in an examination object by way of a magnetic resonance apparatus. A controller ascertains a magnetic resonance sequence comprising at least one sub-sequence for detecting at least one substance to be detected in the examination object as a function of the at least one substance to be detected, and ascertains at least one measuring instant for capturing a respective MRT signal to detect the at least one substance to be detected in the examination object as a function of the at least one substance to be detected. The at least one MRT signal is evaluated for fulfillment of a predetermined detection condition, and a presence of the at least one substance to be detected is established when the at least one MRT signal fulfils the predetermined detection condition.

### CROSS-REFERENCE TO RELATED APPLICATIONS

The present application claims priority to and the benefit of Europe patent application no. EP 22201229.6, filed on Oct. 13, 2022, the contents of which are incorporated herein by reference in their entirety.

### TECHNICAL FIELD

The disclosure relates to a method for detecting a specified substance to be detected in an examination object by way of a magnetic resonance apparatus, a control facility, a magnetic resonance apparatus, a computer program, and an electronically readable data carrier.

### BACKGROUND

Apparatuses for magnetic resonance tomography (MRT) are imaging apparatuses that use a strong external magnetic field to orient the nuclear spins of an object to be examined and to excite them to precession about the corresponding orientation by applying an RF excitation pulse. The precession or the transition of the spins from this excited state into a state with lower energy generates an electromagnetic alternating field as a response, which can be detected as an MR-MRT signal via receiving antennas.

A position encoding can be impressed on the signals with the aid of magnetic gradient fields, and this subsequently allows the received MRT signal to be associated with a volume element of the examination object. The received MRT signal can then be evaluated to provide, for example, an image representation of the examination object.

To excite examination objects, it is provided that the RF excitation pulses and the gradient fields are provided in a magnetic field resonance sequence. The magnetic field resonance sequence can be characterized by sequence parameters, such as a repetition time TR, an echo time TE, an inversion time TI, and a flip angle α. These influence the signal intensity of the received MRT signal from a volume element. This signal intensity also depends on the substances contained in the volume element, however, with the total signal intensity being composed of the signal intensities of the individual substances. The respective signal intensities of the substances depend, inter alia, on the relaxation times T1 and T2 of these substances. By suitable selection of the sequence parameters of the magnetic resonance sequence, the excitation and signal recording can be adjusted in such a way that the signal intensities can be mapped so as to be T1- or T2-weighted, for example.

Magnetic resonance examinations are applied, for example, for imaging to enable a two-dimensional or three-dimensional mapping of the examination object. Different signal intensities are mapped in the mapping, for example with the aid of different gray scale values. The local signal intensity depends, inter alia, on the locally present substances. On the basis of the local signal intensity it is therefore possible, to a certain extent, to identify locally present substances or tissue types.

The aim of clinical capture of MRT recordings almost always consists in converting the physiology or anatomy into items of diagnostic information. This means that on the basis of the MRT recordings, it is possible to identify pathologies and their severity. An interpretation and assessment of image reconstructions by radiologists is necessary in particular in the case of the described imaging, to be able, for example, to establish a presence of particular substances or pathological tissue.

### SUMMARY

Until now, the presence of a particular substance or tissue in the examination object has in most cases been checked by radiologists by way of the reconstruction of images and their assessment. Initial approaches for automatic image analysis have been proposed to automatically identify particular substances, including AI-assisted methods, but images continue to be used as the starting point for detecting particular substances.

A further approach provides an application of spectroscopic methods to be able to detect particular substances or particular tissue and thus assess a malignancy of tissue. Here, the signal intensity of substances is measured with a resonance frequency that is characteristic for them and this is represented, for example, in the form of a resonance frequency-intensity graph. Only items of information from a relatively large volume element may be captured thereby, however. Capture is also relatively slow.

Furthermore, approaches are also known that enable a substance present in the examination object to be ascertained, for example by means of what is known as a magnetic resonance fingerprinting method. To carry out detection of a substance by means of this method, it is provided that signals of the examination object are captured at a plurality of instants to ascertain a characteristic over time of a total signal intensity at an image point. This signal time characteristic is then compared with simulated signal time characteristics, which were generated for the particular assumptions that were specified about the tissue properties (for example T1 and T2 values) during the simulation.

Magnetic resonance fingerprinting methods break the conventional paradigm of data capture by application of regular magnetic sequences of radio-frequency and gradient pulses. To carry out the magnetic resonance fingerprinting methods, the radio-frequency sequence and the gradient sequence of the magnetic resonance sequence are optimized such that the captured, localized image data supplies not just a single image, but rather a series of images. Signal time characteristics are extracted from this series, and these can be aligned with a computer-generated dictionary of simulated signal time characteristics of the signal intensities for the corresponding magnetic resonance sequence on the basis of the different substance parameters of the substances, such as the relaxation times T1, T2 and the chemical shift. By aligning the captured signal time characteristics with known characteristics of signal intensities for respective substance parameters, it is possible to identify these substance parameters, such as T1 or T2. When carrying out the magnetic resonance fingerprinting method, data is captured for magnetic sequences of radio-frequency and gradient pulses that are not conventionally regular by application.

One possible magnetic resonance fingerprinting (MRF) method is disclosed, for example, in DE 10 2019 206 827 A1. In the method, it is provided that parameter values in image points of an examination volume of an examination object are ascertained in an MR system by means of a magnetic resonance fingerprinting technique. At least one image point-time-series of the examination object is carried out in the method with the aid of an MRF recording method of the type that captured image point-time-series are comparable with loaded comparison signal characteristics. A signal of at least one segment of the respective signal characteristic of the captured image point-time-series is compared with a corresponding segment of loaded comparison signal characteristics to ascertain similarity values of the captured image point-time series with the respective comparison signal characteristics.

One drawback of magnetic resonance fingerprinting methods is that the magnetic sequences necessary for it are relatively long to be able to provide an image point-time series.

It is thus an object of the disclosure to provide an MRT method that requires a shorter time for ascertaining the presence of a particular substance in an examination object. This object is achieved by the embodiments and respective subject matter as discussed herein, including the claims.

A first aspect of the disclosure relates to a method for detecting a specified substance to be detected in an examination object by way of a magnetic resonance apparatus.

In a first step, a control facility receives an instruction to detect at least one of the substances potentially present in the examination object. In other words, it is specified to the control facility which potentially present substance is to be checked by the magnetic resonance apparatus for a presence in the examination object.

Implementation of a magnetic resonance examination for detecting the substance in the examination object is accordingly arranged by the control facility.

For this, in one step of the method, the control facility ascertains a magnetic resonance sequence, which comprises at least one sub-sequence, for detecting the at least one substance in the examination object. The at least one sub-sequence can comprise a regular or an irregular sequence of echo and/or gradient sequences. For example, flip angles and/or repetition times can be varied. The at least one sub-sequence can be a time segment of the magnetic resonance sequence. In other words, the control facility generates or retrieves the magnetic resonance sequence, which is to be carried out by the magnetic resonance apparatus, to detect the at least one substance to be detected in the examination object. In other words, the magnetic resonance sequence is generated by the control facility as a function of the at least one substance to be detected in the examination object. In accordance with the substance to be detected, the magnetic resonance sequence is ascertained which is suitable for capturing the substance to be detected in the examination object. It can be provided, for example, that a simulated or stored characteristic of a signal intensity for the substance to be detected is taken into account when ascertaining the magnetic resonance sequence. The magnetic resonance sequence can comprise excitation times and measurement times.

It is provided that the control facility ascertains at least one measuring instant in the magnetic resonance sequence for capturing a respective MRT signal to detect the at least one substance to be detected in the examination object. The at least one measuring instant can be an echo time of the magnetic resonance sequence at which the respective MRT signal is to be captured. The control facility ascertains at least one measuring instant as a function of the at least one substance to be detected. In other words, the control facility ascertains the at least one measuring instant at which the respective MRT signal is to be captured by the magnetic resonance apparatus to enable the presence of the at least one substance to be detected in the examination object. The MRT signal can have a total signal intensity, which can be composed of signal intensities of substances of the examination object. The at least one measuring instant can be, for example, an echo instant of the magnetic resonance sequence, or a selected instant of the magnetic resonance sequence at which the MRT signals can be captured particularly effectively. The at least one measuring instant is likewise ascertained by taking into account the at least one substance to be detected.

After ascertaining the magnetic resonance sequence and the at least one measuring instant, the magnetic resonance apparatus is actuated by the control facility to provide the ascertained magnetic resonance sequence by way of the magnetic resonance apparatus.

In a subsequent step, the magnetic resonance apparatus is actuated for capturing the at least one MRT signal of the examination object to detect substances at the at least one measuring instant in the magnetic resonance sequence. The at least one measuring instant can be, for example, the echo instant or a selected instant in the magnetic resonance sequence at which the MRT signals can be captured particularly effectively.

In a subsequent step, the control facility receives the at least one MRT signal and a presence of the at least one substance to be detected is established when the at least one MRT signal fulfills a predetermined detection condition. In other words, the control facility receives the MRT signal captured by the magnetic resonance apparatus. The control facility evaluates the received MRT signal to check whether the substance to be detected is present in the examination object. The control facility establishes the presence of the substance to be detected in the examination object if the at least one MRT signal fulfills the predetermined detection condition. The predetermined detection condition can specify, for example, overshooting of a predetermined threshold value by a predetermined total signal intensity of the at least one MRT signal.

When the predetermined condition is fulfilled, the control facility can initiate a predetermined method step. For example, a detection signal can be output which can indicate the detected substance(s). If the predetermined condition is not fulfilled, an output of another detection signal can be provided, which indicates that none of the substances to be detected could be detected. Initiation of further steps, such as carrying out or adapting a further sequence, can also be provided when detecting the at least one substance to be detected.

The disclosure results in the advantage that for detecting the at least one substance to be detected, a magnetic resonance sequence ascertained for this purpose and a measuring instant ascertained for this purpose are used, which are optimized to the respective substance to be detected.

More efficient detection of a presence of the at least one substance to be detected in the examination object is consequently possible than is the case with substance-independent magnetic sequences. Faster capture of a presence of the substance is consequently possible than is the case, for example, with currently customary fingerprinting methods because a time series of a plurality of MRT signals is always necessary with fingerprinting methods. The core idea of the disclosure consists in that the measurement does not necessarily have to be spatially resolved, whereby measuring time can be reduced compared with fingerprinting methods. The measurement is parameterized in such a way that it is very sensitive to the particular substance to be detected, and further measurements no longer have to be carried out if the substance to be detected is not detected.

The disclosure also comprises developments which result in further advantages.

One development of the disclosure provides that the control facility receives an item of information about at least one further substance, which is potentially present in the examination object. In other words, the control facility receives items of information about which at least one further substance resides, or could reside, in the examination object in addition to the at least one substance to be detected.

The control facility ascertains the magnetic resonance sequence, which comprises the at least one sub-sequence, for detecting the at least one substance to be detected in the examination object as a function of the at least one potentially present further substance. In other words, the control facility ascertains the magnetic resonance sequence by taking into account the at least one potentially present further substance. In other words, during generation of the magnetic resonance sequence, the control facility takes into account which potentially present substance is present in the examination object to ascertain, in accordance with the potentially present substance, the magnetic resonance sequence which is suitable for capturing the substance to be detected in the presence of the at least one further substance of the examination object. For example, it is possible to take into account how, apart from the substance to be detected, the at least one further substance is excited by the magnetic resonance sequence. It can be provided, for example, that a characteristic of a signal intensity for the further substance is taken into account as a function of the magnetic resonance sequence.

It is provided that the control facility ascertains at least one measuring instant for capturing a respective MRT signal to detect the at least one substance to be detected in the examination object. The control facility ascertains at least one measuring instant as a function of the at least one substance to be detected and the potentially present further substance. In other words, the control facility ascertains the at least one measuring instant at which the respective MRT signal is to be captured by the magnetic resonance apparatus to enable the presence of the at least one substance to be detected in the examination object. The MRT signal can have a total signal intensity, which can be composed of the signal intensities of the substance to be detected and of the at least one further substance. The at least one measuring instant can be, for example, an echo time of the magnetic resonance sequence. The at least one measuring instant is likewise ascertained by taking into account the substance to be detected and the at least one further substance.

One development of the disclosure provides that the sub-sequence for detecting the at least one substance to be detected in the examination object is ascertained according to a predetermined sequence-generating algorithm as a function of substance parameters of the at least one substance to be detected and/or of the at least one potentially present further substance. It is provided that the predetermined sequence-generating algorithm is configured to generate the sub-sequence in accordance with a specified optimization criterion. The specified optimization criterion can specify that the sub-sequence is to be optimized by the sequence-generating algorithm to the extent, that a characteristic of a signal intensity of the MRT signal for the at least one substance to be detected fulfills a predetermined distinguishing criterion in relation to a respective characteristic of a signal intensity of the MRT signal for the at least one potentially present further substance. In other words, it is provided that according to the predetermined sequence-generating algorithm, the control facility ascertains the sub-sequence for detecting the at least one substance to be detected. The sequence-generating algorithm can comprise, for example, a mathematical function, a model, or a simulation, which are provided to ascertain the sub-sequence as a function of the substance parameters of the potentially present substances. The substance parameters of the potentially present substances can comprise, for example, a T1, a T2, or a T2* relaxation time, or also parameters such as Apparent Diffusion Coefficient (ADC) or off resonance. In other words, the substance parameters are input variables of the predetermined sequence-generating algorithm. The predetermined sequence-generating algorithm can be provided to generate the sub-sequence as a function of the substance parameters in such a way that a characteristic of the signal intensity of the MRT signal for the at least one substance to be detected fulfils a predetermined distinguishing criterion to enable detection of the substance in the MRT signal. The distinguishing criterion can comprise, for example, specifications in relation to the characteristics of the signal intensities and/or individual instants of the characteristics. The function of the sequence-generating algorithm can be, for example, a maximization function. The sequence-generating algorithm can be configured, for example, to provide a sub-sequence for an optimally high signal intensity and/or an optimally high signal intensity component of the signal intensity of the at least one substance to be detected in a total signal intensity. The sequence-generating algorithm can also be configured to ascertain the sub-sequence, which causes signal characteristics for the substances, which may be optimally differentiated from one another.

One development of disclosure provides that the predetermined optimization criterion specifies a maximization of a signal intensity of the MRT signal for the at least one substance to be detected at one instant at least of the magnetic resonance sequence. In other words, it is provided that the predetermined optimization criterion specifies a provision of a sub-sequence, which causes a maximum signal intensity of the MRT signal of the substance to be detected at one instant at least of the magnetic resonance sequence. In other words, the optimization criterion stipulates that the characteristic of the signal intensity of the MRT signal of the substance to be detected at one instant at least of the magnetic resonance sequence has an optimally high maximum.

One development of the disclosure provides that the predetermined optimization criterion specifies a minimization of a signal intensity of the MRT signal for the at least one potentially present further substance at one instant at least of the magnetic resonance sequence. In other words, it is provided that the predetermined optimization criterion specifies a provision of a sub-sequence, which causes a minimum signal intensity of the MRT signal of the potentially present further substance at one instant at least of the magnetic resonance sequence.

One development of the disclosure provides that the predetermined optimization criterion specifies a maximization of a signal component of the signal intensity in a total signal intensity of the MRT signal for the at least one substance to be detected at one instant at least of the magnetic resonance sequence. In other words, the optimization criterion specifies that a signal component of the signal intensity of the substance to be detected has a maximum at one instant at least of the magnetic resonance sequence. In other words, the magnetic resonance sequence is to be optimized to the extent that the characteristics of the respective signal intensities of the respective substances proceed in such a way that the signal intensity of the MRT signal of the at least one substance to be detected at one instant at least of the magnetic resonance sequence has a maximum component in a total intensity. The aim is that at one instant at least of the magnetic resonance sequence the total signal intensity matches the signal intensity of the at least one substance to be detected. In other words, it is provided that the signal intensities of the further substances are suppressed at the at least one instant of the magnetic resonance sequence.

One development of the disclosure provides that the predetermined optimization criterion specifies a minimization of a signal component of the signal intensity in a total signal intensity of the MRT signal for the at least one potentially present further substance at one instant at least of the magnetic resonance sequence. In other words, the optimization criterion specifies that a signal component of the signal intensity of the potentially present further substance has a minimum at one instant at least of the magnetic resonance sequence. In other words, the magnetic resonance sequence is to be optimized to the extent that the characteristics of the respective signal intensities of the respective substances proceed in such a way that the signal intensity of the MRT signal of the at least one potentially present further substance has a minimum component in a total intensity at one instant at least of the magnetic resonance sequence.

One development of the disclosure provides that the control facility ascertains at least two possible sub-sequences for detecting the respective substance for the at least one substance to be detected. In other words, at least two possible sub-sequences respectively are generated by the control facility and/or retrieved from a database for the respective substances to be detected. The possible sub-sequences can have been generated, for example, according to respective different algorithms or be retrieved from different libraries and thus differ from one another. The sub-sequences can be provided, for instance, for the present substances and/or for detecting the at least one substance to be detected. At least two sub-sequences can consequently be provided, which are suitable for detecting the at least one substance to be detected. It can accordingly be provided that those of the possible sub-sequences which are more suitable for the measurement are identified and selected. In a further step, it is provided that for the at least two possible sub-sequences the control facility ascertains respective characteristics of respective signal intensities of the MRT signal for the respective substances over a time of the possible sub-sequences. In other words, the control facility ascertains which characteristics respective signal intensities have, which can be attributed to the respective substances. The characteristics can be, for example, calculated, simulated, or retrieved. In a further step, the control facility can select one of the at least two possible sub-sequences as a function of at least one of the signal characteristics according to a predetermined selection criterion as a sub-sequence for detecting the at least one substance to be detected. In other words, it is provided that the control facility selects one of the at least two possible sub-sequences for detecting the respective substance in the examination object. The selection is made according to a predetermined selection criterion. The selection criterion can specify which of the possible sub-sequences is selected, and be optimized to the respective measurement or the respective substance to be detected. The predetermined selection criterion can be based, for example, on the signal characteristic of the signal intensity of the MRT signal for the substance to be detected or the signal characteristics of the signal intensities of all substances.

One development of the disclosure provides that the control facility ascertains for the respective possible sub-sequences a respective maximum value of a signal intensity component of the signal intensity of the MRT signal for the at least one substance to be detected in a total signal intensity of the MRT signal, with the total intensity of the MRT signal being composed of the signal intensities of the respective substances contained in the volume element and contributing to the signal. The selection criterion specifies that that one of the possible sub-sequences which has the greatest maximum value of the signal intensity component of the substance to be detected is selected as the sub-sequence. In other words, the possible sub-sequence which causes a greatest maximum value of the signal intensity component of the substance to be detected is selected as the sub-sequence for detecting the respective substance to be detected. In other words, the respective signal intensity components, which have the individual substances at one instant at least, are ascertained for the respective possible sub-sequences. It can be provided that the respective signal intensity component is ascertained over a total time of the respective sub-sequences, at a plurality of random or particular instants of the respective sub-sequences or at a single instant. The total intensity of the MRT signal can be composed of the signal intensities of the potentially present substances. For each of the possible sub-sequences, it is possible to ascertain the respective maximum value, which the signal intensity component of the substance to be detected has in the respective possible sub-sequence. The selection criterion can provide that the sub-sequence whose maximum value of the signal intensity component of the substance to be detected is greatest is selected. This results in the advantage that the sub-sequence that has a maximum intensity component of the substance to be detected is selected.

One development of the disclosure provides that respective characteristics of respective signal intensities of the MRT signal for the respective substances are ascertained for the magnetic resonance sequence over a time of the ascertained magnetic resonance sequence. In a further step, at least one instant of the time is ascertained at which a signal intensity component of the signal intensity of the substance to be detected respectively has a maximum in a total signal intensity of the MRT signal. The total intensity of the MRT signal is composed of the signal intensities of the respective substances. It is provided that the at least one instant at which the signal intensity component of the signal intensity of the substance to be detected respectively has a maximum is ascertained as the measuring instant for capturing the respective MRT signal to detect the respective substance in the examination object. In other words, it is provided that the control facility ascertains the instant at which the signal intensity component provided by the substance to be detected has a maximum during the magnetic resonance sequence. The control facility defines the ascertained instant in the magnetic resonance sequence as the measuring instant for detecting the substance to be detected respectively. The development results in the advantage that a purposefully ascertained instant is selected for detecting the presence of the substance to be detected respectively.

One development of the disclosure provides that the control facility receives an instruction for detecting at least two of the substances potentially present in the examination object. In other words, it is provided that the control facility receives an instruction, which instructs the control facility to check at least two of the potentially present substances for a presence in the examination object. It can be provided, for example, that at least two of the at least two substances present in the examination object have to be detected, or more than two potentially present substances.

It is provided that the control facility ascertains at least one respective measuring instant of the magnetic resonance sequence for each of the substances to be detected. In other words, it is provided that for detecting the respective substances, the control facility ascertains respective measuring instants at which the magnetic resonance apparatus captures the respective signals, with a respective instant being determined for detecting the respective substance in the examination object. It can be provided, for example, that the magnetic resonance sequence generates signal characteristics for the respective substances to be detected. A signal intensity component of a first substance can have a maximum at a first instant. A signal intensity component of a second substance can have a maximum at a second instant. In this case, the instant at which the signal intensity component of the first substance has the maximum can be determined as the measuring instant for capturing the MRT signal for detecting the first substance. Analogously, the control facility can determine the instant at which the signal intensity component of the second substance has the maximum as the measuring instant for capturing the MRT signal to detect the second substance by way of the control facility.

One development of the disclosure provides that the control facility ascertains respective sub-sequences for detecting the at least two substances to be detected in the examination object. In other words, it is provided that the magnetic resonance sequence has at least two of the sub-sequences, with the respective sub-sequences being optimized for detecting the substance to be detected, respectively. The development results in the advantage that optimized sub-sequences are provided respectively for detecting the respective substances.

One development of the disclosure provides that the control facility ascertains at least two measuring instants during the magnetic resonance sequence, and the magnetic resonance apparatus captures respective signal characteristics. In other words, it is provided that instead of ascertaining a signal intensity of an MRT signal at a respective measuring instant, a signal characteristic is ascertained. In a further step, it is provided that the control facility reconstructs the characteristic of the signal intensity. The control facility compares characteristic of the signal intensity with predetermined signal characteristics, with the predetermined signal characteristics being associated with respective substances. The detection condition provides fulfillment of a concordance criterion of the captured signal characteristic with one of the predetermined signal characteristics of the respective substance. The presence of the respective substance is confirmed or denied as a function of a measure of concordance between the captured signal characteristic and one of the predetermined signal characteristics. In other words, it is provided that the presence of the at least one substance to be detected is checked by means an analysis of the signal intensity characteristic. This can be, for example, what is known as a fingerprinting method for capturing the presence of the respective substance by way of the control facility. In contrast to customary fingerprinting methods, however, a magnetic resonance sequence optimized to the substance to be detected is provided.

One development of the disclosure provides that the magnetic resonance sequence comprises at least one spatially encoded sequence segment, and the control facility ascertains a position of the at least one substance to be detected in the examination object by means of the at least one spatially encoded sequence segment. In other words, the magnetic resonance sequence is designed in such a way that it prompts a spatial encoding of the MRT signal. In other words, the magnetic resonance sequence is designed in such a way that the control facility can associate the respective MRT signal with a respective location in the examination object. The magnetic resonance sequence can comprise, for example, a gradient echo sequence. This results in the advantage that, in addition to the presence of the substance to be detected in the examination object, its position in the examination object can be ascertained. The spatial encoding is not imperative, however. It can be provided that it only takes place when the at least one substance to be detected is detected.

One development of the disclosure provides that the position of the at least one substance to be detected is merged with an image representation of the examination object. It can be provided, for example, that the magnetic resonance sequence comprises an image-capturing sequence for capturing image data. The control facility can reconstruct the image representation of the examination object from the image data. The control facility can also receive the image representation. The positions at which the at least one substance to be detected is captured can be highlighted in color in the image representation. It can also be provided that the control facility can identify structures present in the image representation as the at least one substance to be detected. In this case, for example an enclosed area of a similar shade of gray, can be associated with the substance. This results in the advantage that image representations can be evaluated by taking into account detected substances. A spatial resolution when detecting the position of the substance can also be lower than a spatial resolution of the image representation.

One development of the disclosure provides that the control facility ascertains a quantitative variable for the at least one substance to be detected. The quantitative variable can relate, for example, to a probability of a presence of the substance in the examination object or at least the one volume element, to a proportion of the substance or an absolute quantity of the substance. This results in the advantage that a quantification of the substance is made possible. The quantification can relate to the object as a whole and/or be location-dependent. A composition can be ascertained when a plurality of substances is detected. The quantitative variable can be obtained as a reference in relation to one of the substances.

For specific applications, or application situations which can result with the method and which are not explicitly described here, it can be provided that according to the method, an error message and/or a request to input user feedback is output and/or a standard setting and/or a predetermined initial state is set.

A second aspect of the disclosure relates to a control facility. The control facility is configured to actuate a magnetic resonance apparatus to detect a specified substance in an examination object. The control facility is configured to receive an instruction to detect at least one substance to be detected. To enable a presence of the at least one substance to be detected in the examination object, the control facility is configured to ascertain a magnetic resonance sequence comprising at least one sub-sequence for detecting the at least one substance to be detected in the examination object. The control facility is configured to ascertain at least one measuring instant for capturing a respective MRT signal to detect the at least one substance to be detected in the examination object. The control facility is configured to actuate the magnetic resonance apparatus for providing the ascertained magnetic resonance sequence, and to actuate the magnetic resonance apparatus for capturing the MRT signal of the examination object at the at least one measuring instant. The control facility is configured to receive the at least one MRT signal, and to establish a presence of the at least one substance to be detected when the at least one MRT signal fulfils a predetermined condition.

A control facility can be taken to mean, for example, any suitable type of data processing device that contains a processing circuit and/or one or more processors. The control facility can thus process, for instance, data for carrying out computing operations. These optionally also include operations to carry out indicated instances of access to a data structure, for example a look-up table (LUT).

The control facility may also be referred to herein as a computing unit, a control device, a control computer, or a controller, and may include, for example, one or more computer(s), one or more microcontroller(s), and/or one or more integrated circuit(s), for example one or more Application-Specific Integrated Circuits (ASIC), one or more Field Programmable Gate Arrays (FPGA), and/or one or more System(s) on a Chip (SoC). The control facility can also include one or more processor(s), for example one or more microprocessor(s), one or more Central Processing Unit(s) (CPU), one or more Graphics Processing Unit(s) (GPU) and/or one or more signal processor(s), in particular one or more Digital Signal Processor(s) (DSP). The control facility can also include a physical or a virtual group of computers or other of said units.

A third aspect of the disclosure relates to a magnetic resonance apparatus, which has at least one control facility.

A fourth aspect of the disclosure relates to a computer program product. Furthermore, a computer program product can be provided, which can be loaded directly into a memory of a control facility of a magnetic resonance tomography system, with program means to carry out the steps of any of the methods described herein when the program is executed in the control facility of the magnetic resonance tomography system. The method described herein can also be in in the form of a computer program product, which implements any of the methods described herein on a control facility when it is executed on the control facility.

A fifth aspect of the disclosure relates to an electronically readable data carrier.

Similarly, an electronically readable data carrier with electronically readable items of control information stored thereon can be present, which comprise at least one described computer program product and are configured in such a way that they carry out any of the methods described herein when the data carrier is used in a control facility of an MR system.

### BRIEF DESCRIPTION OF THE DRAWINGS

Further advantages, features, and details of the disclosure will emerge from the exemplary embodiments described below and from the associated drawings in which:

FIG. 1 illustrates a schematic representation of an embodiment of an example magnetic resonance apparatus;

FIG. 2 illustrates a schematic representation of a sequence of an embodiment of an example method;

FIG. 3 illustrates a schematic representation of an example magnetic resonance sequence; and

FIG. 4 illustrates a schematic representation of an example image mapping of an examination object with localized substances.

### DETAILED DESCRIPTION OF THE DISCLOSURE

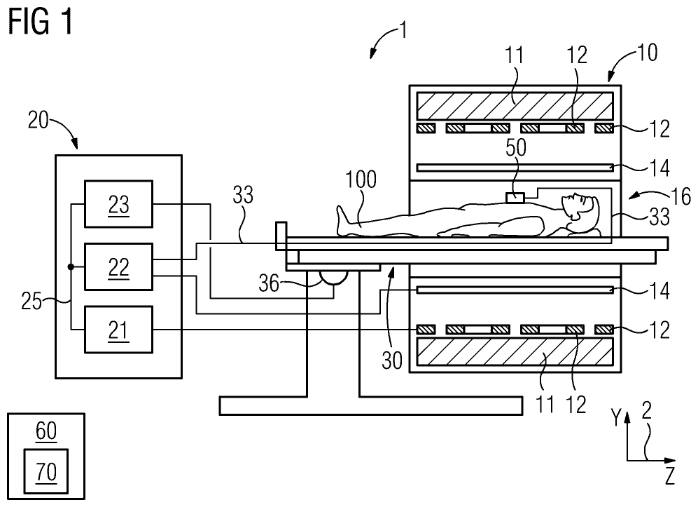

FIG. 1 illustrates a schematic representation of an embodiment of an example magnetic resonance apparatus. The magnetic resonance apparatus 1 has a magnetic unit 10 with a field magnet also referred to herein as a main magnet) 11, which generates a static magnetic field for orienting nuclear spins of a sample, for example of an examination object 100, in an examination region or recording region. The recording region is characterized by an extremely homogeneous static magnetic field, with the homogeneity relating, in particular, to the magnetic field strength or its value. The recording region is, for example, almost spherical and is positioned in a patient tunnel 16, which extends through the magnetic unit 10 in a longitudinal direction 2. The field magnet 11 can be, for example, a superconducting magnet, which can provide magnetic fields with a magnetic flux density of up to 3 T or more. Permanent magnets or electromagnets with normal-conducting coils can also be used for lower field strengths, however. A patient couch 30 can be moved in the patient tunnel 16 by a traversing unit 36.

Furthermore, the magnetic unit 10 has gradient coils 12, which for spatial differentiation of the captured mapping regions in the recording region are designed for overlaying the static magnetic field with location-dependent magnetic fields in the three spatial directions. The gradient coils 12 can be embodied, for example, as coils made of normal-conducting wires, which can generate, for example, mutually orthogonal fields or field gradients in the recording region.

The magnetic unit 10 can have, for example, a body coil 14 as a transmitting antenna, which coil is designed to radiate a radio-frequency signal supplied via a signal line into the examination region. In some embodiments, the body coil 14 can also be used to receive resonance signals emitted by the examination object 100 and to emit them via a signal line. In such embodiments, the body coil 14 can serve as a main receiving antenna as well as a transmitting antenna. In addition, specific coils adapted for particular organs (what are known as local coils) can also be used for receiving the resonance signals.

The magnetic resonance apparatus 1 has a control facility 20, which again may also be referred to herein as a computing unit, a control device, a control computer, or a controller, and which may supply the magnetic unit 10 with different signals for the gradient coils 12 and the body coil 14 and which can evaluate received signals. The control facility 20 can have, for example, a gradient controller 21, which is configured to supply the gradient coils 12 with variable currents via supply lines, which currents can provide the desired gradient fields in the examination region in a time-coordinated manner.

The control facility 20 can also have a radio-frequency unit (also referred to herein as RF circuitry) 22, which is configured to generate radio-frequency pulses or excitation pulses with specified characteristics over time, amplitudes, and spectral power distribution for exciting a magnetic resonance of the nuclear spins in the examination object 100. Pulse powers in the region of kilowatts can be used in this case. The excitation pulses can be radiated into the examination object 100 via the body coil 14 or via one or more local transmitting antenna(s). The control facility 20 can also include a controller 23, which can communicate with the gradient controller 21 and the radio-frequency unit 22 via a signal bus 25.

Optionally, a local coil 50 can be arranged in the immediate vicinity of the examination object) 100, for example on the examination object 100 or in the patient couch 30, which coil can be connected to the radio-frequency unit 22 via a connecting line 33. Depending on the embodiment, the local coil 50 can serve, as an alternative or in addition to the body coil 14, as a main receiving antenna.

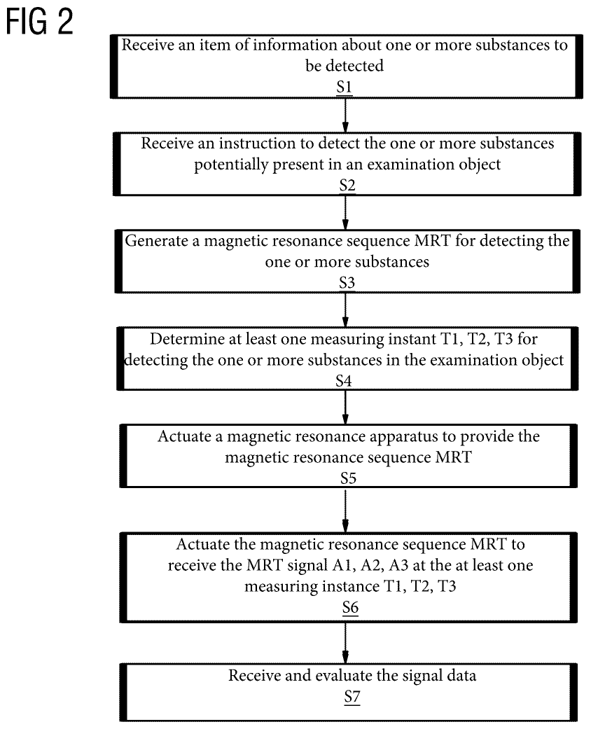

FIG. 2 illustrates a schematic representation of a sequence of an embodiment of an example method. FIG. 2 shows an exemplary embodiment of a sequence of a method for detecting a specified substance in an examination object 100 by way of a magnetic resonance apparatus 1.

In a first block S1, a control facility 20 can receive an item of information about at least one substance to be detected, which is potentially present in the examination object 100. The information can also comprise at least one potentially present further substance. The information about the potentially present substance(s) can also be based on prior knowledge and be based, for example, on known tissue types in the brain/head. The prior knowledge can be stored in the control facility 20 in this case. The examination object 100 can be a body composed of inorganic and/or organic material. The examination object 100 can be an animate being, e.g. a human. The examination object 100 can have a plurality of substances. It can be the case that the substances present in the examination object 100 and/or their positions in the object can be unknown. To detect a presence of at least one of the substances, the control facility 20 can be supplied with the information about which substances potentially present in the examination object 100 are potentially present. The substances can be described by substance parameters of the respective substances, such as, for example, relaxation parameters T1, T2, T2* of the respective materials. The control facility 20 can ascertain, for example for the respective substance parameters, which characteristic a signal intensity B1, B2, B3 of an MRT signal A1, A2, A3 has over time if the respective substance with a magnetic resonance sequence MRT is excited by a magnetic resonance apparatus 1. The characteristic can be ascertained, for example, according to a formula or a simulation.

In a second block S2, the control facility 20 can receive an instruction to detect at least one of the substances potentially present in the examination object 100. The instruction can prompt the control facility 20 to actuate the magnetic resonance apparatus 1 to detect at least one of the substances in the examination object 100. The control facility 20 is thus instructed to actuate the magnetic resonance apparatus 1 in such a way that it provides a magnetic resonance sequence MRT for exciting the examination object 100 to capture at least one MRT signal A1, A2, A3 of the examination object 100, which is suitable for detecting the at least one substance to be detected in the examination object 100.

In a block S3, the control facility 20 can generate the magnetic resonance sequence MRT for detecting the at least one substance to be detected according to a sequence-generating algorithm. The magnetic resonance sequence MRT can have one or more sub-sequence(s) MRT1, MRT2, which follow one another. The control facility 20 can ascertain, for example, sequence parameters, such as repetition times, the inversion times, or flip angles of the sub-sequences MRT1, MRT2. The sequence-generating algorithm can provide a formula, a model, or a simulation for capturing the magnetic resonance sequence MRT. The sequence-generating algorithm can be provided to optimize a magnetic resonance sequence MRT to the extent that the at least one substance can be detected in the examination object 100 by way of the at least one MRT signal A1, A2, A3. Ascertaining can take place as a function of the at least two potentially present substances. A plurality of sub-sequences MRT1, MRT2 can also be ascertained by the sequence-generating algorithm to be able to detect the respective substances to be detected.

In a block S4, the control facility 20 can ascertain at least one measuring instant T1, T2, T3 for capturing a respective MRT signal A1, A2, A3 for detecting the at least one substance to be detected in the examination object 100 as a function of the at least one substance to be detected solely or additionally as a function of the at least one potentially present further substance. In other words, the control facility 20 ascertains a measuring instant T1, T2, T3 in order to be able to capture a suitable MRT signal A1, A2, A3 for detecting the at least one substance to be detected. The at least one measuring instant T1, T2, T3 can be ascertained as a function of respective characteristics of the signal intensities B1, B2, B3 of the respective substances. The characteristics of the signal intensities B1, B2, B3 of the respective substances can be ascertained, for example, from substance parameters of the substances by means of simulations or formulae. The control facility 20 can select, for example, the measuring instant T1, T2, T3 in such a way that in the case of the substance parameters of the at least one substance to be detected and the substance parameters of the at least one potentially present further substance, it can be assumed that an MRT signal A1, A2, A3 captured at the measuring instant T1, T2, T3 has a total intensity, which is based solely or for the most part on a signal intensity B1, B2, B3 of the at least one substance to be detected.

In a block S5, the control facility 20 can actuate the magnetic resonance apparatus 1 so it provides the magnetic resonance sequence MRT.

In a block S6, the control facility 20 can actuate the magnetic resonance sequence MRT so it receives the MRT signal A1, A2, A3 at the at least one measuring instant T1, T2, T3.

Items of information, such as a total signal intensity B of the MRT signal A1, A2, A3, can be sent to the control facility 20 by the magnetic resonance sequence MRT.

In a block S7, the control facility 20 can receive and evaluate the signal data. In the evaluation, it is possible to check at least whether the at least one substance to be detected is present in the examination object 100. The presence of the at least one substance to be detected can be checked by checking fulfillment of a specified detection condition. The detection condition can provide, for example, overshooting of a specified threshold value by the total intensity B of the MRT signal A1, A2, A3. If the detection condition is fulfilled, the control facility 20 can establish the presence of the at least one substance to be detected and output a predefined output signal. If the detection condition is not fulfilled, output of a different output signal can be provided.

In the case of capture of the at least one substance to be detected, further steps to be carried out additionally or alternatively by the control facility 20 can be provided, such as, for example, ascertaining a quantitative variable in relation to the at least one substance to be detected, which can indicate a quantity, density or probability of presence of the substance in the examination object 100 or at least one volume element. The quantitative variable can be ascertained, for example, as a function of a signal intensity B1 of the substance to be detected alone or in comparison with a signal intensity B2 of a further one of the substances. The MRT signal A1 received in block S6 can e.g. be used for ascertaining the quantitative variable. The quantitative variable can be calculated, for example, from the signal intensity according to a predetermined method. A highly resolved image recording could be triggered here.

A fusion with image data of the object can also be provided. In this case, an additional sequence for localization can be provided if this has not yet taken place in the magnetic resonance sequence MRT.

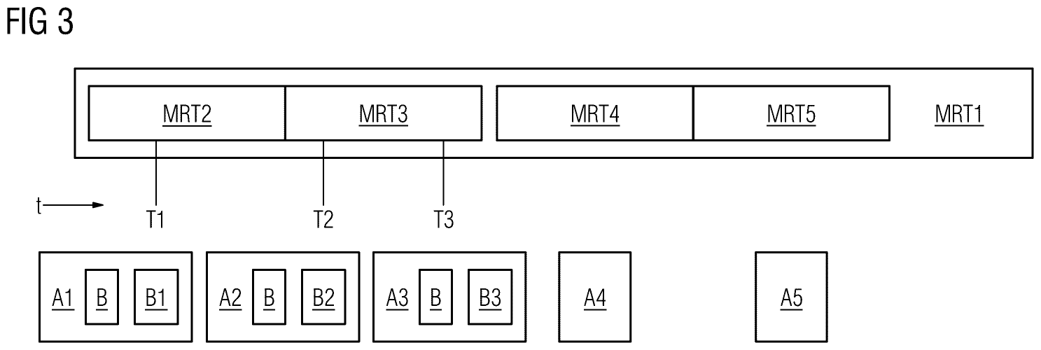

FIG. 3 illustrates a schematic representation of an example magnetic resonance sequence. Specifically, FIG. 3 shows a schematic representation of a magnetic resonance sequence MRT.

The magnetic resonance sequence MRT can be generated according to the predetermined algorithm and have two sub-sequences MRT1, MRT2. The sub-sequences MRT1, MRT2 can be provided to enable detection of substances of the examination object 100. A first sub-sequence can be provided for detecting a first one of the substances. For detecting the first substance, a first measuring instant T1, T2, T3 can be provided at which a first MRT signal A1, A2, A3 is captured by the magnetic resonance apparatus 1. The first measuring instant T1, T2, T3 can be selected in such a way that a total signal intensity B of the first MRT signal A1, A2, A3 is identical to a signal intensity B1, B2, B3 of the first substance. It can consequently be possible to check fulfillment of the detection condition by the signal intensity B1, B2, B3 of the first substance.

A second sub-sequence can be provided for detecting a second and a third one of the substances. For detecting the second substance, a second measuring instant T1, T2, T3 can be provided at which a second MRT signal A1, A2, A3 is captured by the magnetic resonance apparatus 1. The second measuring instant T1, T2, T3 can be selected in such a way that a total signal intensity B of the second MRT signal A1, A2, A3 is identical to a signal intensity B1, B2, B3 of the second substance. It can consequently be possible to check fulfillment of the detection condition by the signal intensity B1, B2, B3 of the second substance.

For detecting the third substance, a third measuring instant T1, T2, T3 can be provided at which a third MRT signal A1, A2, A3 is captured by the magnetic resonance apparatus 1. The third measuring instant T1, T2, T3 can be selected in such a way that a total signal intensity B of the third MRT signal A1, A2, A3 is identical to a signal intensity B1, B2, B3 of the third substance. It can consequently be possible to check fulfillment of the detection condition by the signal intensity B1, B2, B3 of the third substance.

It can be provided that a localization sequence MRT3 is provided when detecting at least one of the substances, which sequence can be provided to supply localization signals A4, which enables localization of at least one of the substances in the examination object 100. The localization sequence MRT3 can comprise, for example, a gradient echo sequence.

It can be provided that the magnetic resonance sequence MRT comprises an image-capturing sequence MRT4, which can be provided to supply image capture signals A5, which enable generation of an object mapping of the examination object 100 by the control facility 20.

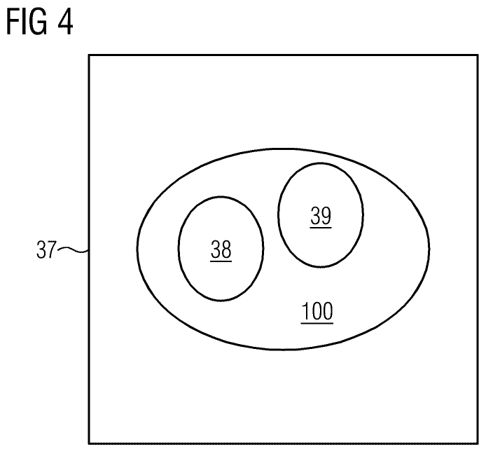

FIG. 4 illustrates a schematic representation of an example image mapping of an examination object with localized substances. Specifically, FIG. 4 shows a schematic representation of an object mapping 37 of an examination object 100 with localized substances.

The object mapping 37 can be a two-dimensional or three-dimensional mapping of the examination object 100 (e.g. a patient). Substance distributions 38, 39 of respective substances to be detected can be displayed in the object mapping 37.

The disclosure is based on the idea of ascertaining magnetic resonance sequences MRT of RF pulses and gradient pulses, which at particular sampling instants result in signals which can be associated with only one specific substance and/or tissue. An examination object 100 can consequently be confirmed or denied, for example for existence of a substance to be detected, for example a carcinogenic tissue or lesions, without imaging. The method could also be used for identifying and/or determining particular tissue or benign or malignant changes.

In contrast to the general MRT fingerprinting method, it is provided that a magnetic resonance sequence MRT is ascertained, which can be generated individually for capturing the substance to be detected. It can consequently be possible to use the magnetic resonance sequence MRT for carrying out a rapid screening method. The magnetic resonance sequence MRT can be generated according to a predetermined algorithm and can then be optimized such that the magnetic resonance sequence MRT structures an RF sequence and/or a gradient sequence of the magnetic resonance sequence MRT such that the MRT signal A1, A2, A3 has a total signal intensity B, which solely comprises a signal intensity B1, B2, B3, which can be attributed to the substance to be detected.

This can be achieved in that the magnetic resonance sequence MRT is generated on the basis of substance-specific parameter combinations. The substance-specific parameters can comprise the relaxation values T1 and T2, T2* and/or additional parameters, such as the Apparent Diffusion Coefficient (ADC). The RF sequence and/or the gradient sequence of the magnetic resonance sequence MRT can be optimized by the algorithm such that a signal intensity B1, B2, B3 of the substance to be detected is maximized for these parameter combinations. The magnetic resonance sequence MRT can also be optimized by the algorithm to the extent that the signal intensity B1, B2, B3 of the substance to be detected is maximized and the signal intensity B1, B2, B3 of further possible substances of the examination object 100 is minimized. It can consequently be possible to maximize a signal intensity component of the signal intensity B1, B2, B3 of the substance to be detected in a total signal intensity B, which can comprise the signal intensity B1, B2, B3 of all substances of the examination object 100. The algorithm can be based on a function, a model, a table, and/or a simulation.

The method can be directed towards solely establishing a presence of the substance to be detected in the examination object 100. In this case, it can be provided that the method is carried out in such a way that the substance to be detected is not localized or is only relatively roughly localized. The magnetic resonance sequence MRT can be configured in this case, for example, for targeted excitation of the prostate region. The question as to whether and how much of the substance to be detected is present in the target region of the examination object 100 can be answered directly on the basis of capture of an MRT signal A1, A2, A3 whose signal intensity B1, B2, B3 can be attributed to the substance to be detected. The procedure would be comparable in this case to a single-voxel spectroscopy approach, in which the existence of particular metabolites in a large voxel is detected. In the case where no relevant signal intensity B1, B2, B3 is present, it can be assumed that the substance to be detected is not present. The control facility 20 can then output a corresponding output signal. No further measurements are necessary for this case.

The presence of the substance to be detected can be established if a predetermined detection condition is fulfilled, for example the signal intensity B1, B2, B3 overshoots a predetermined threshold value. The control facility 20 can ascertain and output a quantitative value as a function of the signal intensity value, which value can describe, for example, a probability of the existence of the substance to be detected, a proportion of the substance or a quantity of the substance to be detected in the examination object 100 or the region.

In this case, it can be provided that a more accurate position can be ascertained for ascertaining the position of the at least one substance to be detected. For this, the control facility 20 can supplement the magnetic resonance sequence MRT with a sequence segment, which enables a localization of the at least one substance to be detected in the examination object 100, and actuate the magnetic resonance apparatus 1 to provide the additional sequence. This can be enabled, for example, via a gradient echo sequence. Captured signals can be transferred to the control facility 20, which can ascertain the position of the at least one substance to be detected therefrom.

It can also be provided that data about the ascertained position is merged with image recordings of the examination object 100. The control facility 20 can be provided with the image recordings. It can also be provided that the control facility 20 actuates the magnetic resonance apparatus 1 to carry out an imaging scan of the examination object 100 for capturing a 2D or 3D image analysis. The control facility 20 can analyze the image. The presence or an ascertained position can be merged with the image analysis.

Another partial implementation could use a further signal localization, which can optionally be carried with local coil elements or conventional gradient encoding in a gradient echo sequence of the magnetic resonance sequence MRT, and this results in a localized imaging technique in which the individual position of the at least one substance to be detected in the examination object 100 is also ascertained. The magnetic resonance sequence MRT could comprise a gradient or spin echo sequence from the start for position ascertainment. This is comparable to imaging in Positron Emission Tomography (PET), with a specific tracer. This image can be overlaid on high-resolution MR images of the examination object 100 for the anatomical context.

A further feature could be that the magnetic resonance sequence MRT can be encoded not only for detecting the at least one substance to be detected in the examination object 100, but for two, three, or more substances to be detected. The presence of the substances to be detected can be checked during the magnetic resonance sequence MRT. An MRT signal A1, A2, A3 can be provided at a shared measuring point or at different measuring points for detecting the different substances. It can also be provided that a respective sub-sequence MRT1, MRT2 is ascertained for a respective substance. It can also be provided that a sub-sequence MRT1, MRT2 can be provided for at least two of the substances. The sub-sequences MRT1, MRT2 can follow one another time-wise. It can be provided that the substances to be detected are merged, for example are combined or overlaid, with the image recording in different color codings.

The same can occur in a single recording in which the respective parameter combinations are color-coded. The algorithm can also be configured such that instead of generating a magnetic resonance sequence MRT, which is only suitable for detecting a single substance or a group of substances, because they only supply a signal intensity component in a total intensity of an MRT signal A1, A2, A3, it generates the magnetic resonance sequence MRT such that respective signals can be captured at different measuring instants T1, T2, T3 of the magnetic resonance sequence MRT, the signal intensities B1, B2, B3 of which signals can be associated with a respective substance.

In the case of a plurality of tissues whose presence is checked either in a combined manner or successively, one of these substances to be detected could serve as a reference substance. This could enable better quantification. It could be provided that a magnetic resonance sequence MRT is provided in a method, for which two measuring instants T1, T2 are ascertained. A first of the measuring instants T1 can be selected in such a way that a total signal intensity B of the MRT signal A1 captured at the measuring instant T1 can be attributed solely to the signal intensity B1, for example of fat as the substance to be detected. In other words, this measuring instant T1 is selected such that only fat is captured in the MRT signal A1. A further measuring instant T2 can be laid such that only the signal intensity B2, for example of water, is captured in the total signal intensity B of the MRT signal A2. The intensity of the MRT signal A1, A2, A3 of one of the substances can be used as a quantitative reference. The quantitative variable of a further substance can be related to its signal intensity B1, B2, B3 by way of this reference.

Instead of identifying a presence of a substance on the basis of its signal intensity B1, B2, B3 at one measuring point at least, the method can provide a presence of the substance on the basis of a signal characteristic of the signal intensity B1, B2, B3. The magnetic resonance sequence MRT can be formed in such a way that a characteristic of a signal intensity B1, B2, B3 of a substance to be detected can be easily distinguished from the characteristics of the signal intensities B1, B2, B3 of other substances. In other words, the characteristic of the total intensity comprises the characteristics of the intensities of the substances. Instead of the magnetic resonance sequence MRT being formed such that only the signal intensity B1, B2, B3 of one of the substances to be detected is captured at one measuring instant T1, T2, T3 at least and the signal intensities B1, B2, B3 of other substances are minimized, in this case it is not necessary to suppress particular signal components. The individual characteristics only have to proceed in such a way that they may be captured in the characteristic of the total intensity. An algorithm can be provided for capturing the characteristics. The algorithm can provide, for example, simple pattern matching or an identification of a characteristic by means a neural network. The algorithm is capable of identifying whether the captured characteristic of the total signal intensity B contains the characteristic of the signal intensity B1, B2, B3 of the substance. Generation of a magnetic resonance sequence MRT which is suitable for localization is not required in this case. This approach can be carried out without signal localization in order to obtain an assessment as to whether the scanned volume of the examination object 100 contains the at least one substance.

The approach can also be combined by way of the magnetic resonance sequence MRT with localization techniques, such as gradient encoding, however, to be able to generate a mapping with a charting of the substance to be detected, as has already been mentioned.

The result of the identification can comprise a quantitative variable, for example an item of quantity information or a probability of the substance to be detected.

The sequence optimization of RF and gradient pulses can be carried out either analytically, numerically, or with the aid of simulations. The magnetic resonance sequence MRT can take into account further parameters, such as diffusion weightings and/or magnetization transfers, for the purpose of better signal separation.

These rapid screening scans can be part of automated work flows. If a suspect MRT signal A1, A2, A3 has been found, it can be categorized by the described method in that the magnetic resonance apparatus 1 generates and provides a magnetic resonance sequence MRT for capturing a substance to be detected.

Depending on the result, more time-consuming scans can be selected, which are tailored to the suspected pathology.

Rapid screening could also be used for specific local measurements. Since the examination object 100 is already situated in the magnetic resonance apparatus 1, the screening could in principle be added to every standard MR examination.

In contrast to earlier techniques, including MR fingerprinting, no image recording or analysis would be necessary in the described method. A presence and a quantity of the substance in a volume element of the examination object 100 could be ascertained on the basis of the captured MRT signal A1, A2, A3. This could significantly speed up capture and data analysis.

The various components described herein may be referred to as “units.” Such components may be implemented via any suitable combination of hardware and/or software components as applicable and/or known to achieve their intended respective functionality. This may include mechanical and/or electrical components, processors, processing circuitry, or other suitable hardware components, in addition to or instead of those discussed herein. Such components may be configured to operate independently, or configured to execute instructions or computer programs that are stored on a suitable computer-readable medium. Regardless of the particular implementation, such units, as applicable and relevant, may alternatively be referred to herein as “circuitry,” “controllers,” “processors,” or “processing circuitry,” or alternatively as noted herein.

What is claimed is:

1. A method for detecting a substance in an examination object via a magnetic resonance apparatus, the method comprising:
receiving an instruction to detect the substance in the examination object;
determining a magnetic resonance sequence comprising a sub-sequence for detecting the substance as a function of the substance to be detected;
determining a measuring instant within the magnetic resonance sequence for capturing a respective magnetic resonance sequence signal to detect the substance as a function of the substance to be detected;
actuating the magnetic resonance apparatus to provide the determined magnetic resonance sequence;
actuating the magnetic resonance apparatus to capture the magnetic resonance sequence signal of the examination object at the measuring instant within the magnetic resonance sequence; and
detecting the substance when the magnetic resonance sequence signal fulfills a predetermined detection condition based upon an evaluation of the magnetic resonance sequence signal.

2. The method as claimed in claim 1, further comprising:
receiving an item of information of a further substance that is potentially present in the examination object;
determining the magnetic resonance sequence as a function of the further substance; and
determining the measuring instant for capturing the magnetic resonance sequence signal to detect the substance as a function of the further substance.

3. The method as claimed in claim 2, further comprising:
determining the sub-sequence as a function of substance parameters of the substance and/or substance parameters of the further substance,
wherein the sub-sequence is generated based upon a predetermined optimization criterion.

4. The method as claimed in claim 3, wherein the predetermined optimization criterion specifies a maximization of a signal intensity of the magnetic resonance sequence signal for the substance to be detected at one instant within the magnetic resonance sequence.

5. The method as claimed in claim 3, wherein the predetermined optimization criterion specifies a minimization of a signal intensity of the magnetic resonance sequence signal for the potentially present further substance at one instant within the magnetic resonance sequence.

6. The method as claimed in claim 3, wherein the predetermined optimization criterion specifies a maximization of a signal component of a signal intensity in a total signal intensity of the magnetic resonance sequence signal for the substance to be detected at one instant within the magnetic resonance sequence.

7. The method as claimed in claim 3, wherein the predetermined optimization criterion specifies a minimization of a signal component of a signal intensity in a total signal intensity of the magnetic resonance sequence signal for the further substance at one instant within the magnetic resonance sequence.

8. The method as claimed in claim 1, further comprising:
determining two possible sub-sequences for detecting two respective substances in the examination object;
determining, for each of the two possible sub-sequences, characteristics of respective signal intensities of the magnetic resonance sequence signal for each of the two respective substances over a time of execution of the two possible sub-sequences; and
selecting one of the two possible sub-sequences as a function of the respective signal characteristics according to a predetermined selection criterion as the sub-sequence for detecting the substance.

9. The method as claimed in claim 8, further comprising:
determining, for each one of the two possible sub-sequences, a respective maximum value of a signal intensity component of the signal intensity of the magnetic resonance sequence signal for the respective substance to be detected within a total signal intensity of the magnetic resonance sequence signal,
wherein the total signal intensity comprises signal intensities of the two respective substances, and
wherein the predetermined selection criterion comprises an exhibition of a greatest maximum value of a signal intensity component of the substance to be detected.

10. The method as claimed in claim 1, further comprising:
determining, for the magnetic resonance sequence, characteristics of respective signal intensities of the magnetic resonance sequence signal for respective substances over an executed time of the magnetic resonance sequence;
determining at least one instant of time at which a signal intensity component of the signal intensity of a respective substance to be detected in a total signal intensity of the magnetic resonance sequence signal has a maximum,
wherein the total intensity of the magnetic resonance sequence signal comprises signal intensities of the respective substances to be detected, and
determining the at least one instant of time has the maximum as the measuring instant for capturing the respective magnetic resonance sequence signal to detect the respective substance in the examination object.

11. The method as claimed in claim 1, further comprising:
receiving an instruction to detect two substances in the examination object; and
for each of the two substances to be detected, determining at least one respective measuring instant for capturing the respective magnetic resonance sequence signal to detect the respective substance in the examination object.

12. The method as claimed in claim 11, further comprising:
determining, for each of the two substances to be detected, a respective sub-sequence for capturing the respective magnetic resonance sequence signal to detect each respective one of the two substances in the examination object.

13. The method as claimed in claim 1, further comprising:
capturing respective total signal intensities of the magnetic resonance sequence signals at two measuring instants during the magnetic resonance sequence;
determining a characteristic of the total signal intensity from the total signal intensities of the two measuring instants; and
comparing a characteristic of the total signal intensity with a predetermined signal characteristic,
wherein the predetermined signal characteristic is associated with the substance to be detected, and
wherein the predetermined detection condition comprises fulfilling a compliance criterion of the captured characteristic of the total signal intensity in relation to the predetermined signal characteristic of the substance to be detected.

14. The method as claimed in claim 1, wherein the magnetic resonance sequence comprises at least one spatially encoded sequence segment, and further comprising:
determining a position of the substance to be detected in the examination object based upon the at least one spatially encoded sequence segment.

15. The method as claimed in claim 14, further comprising:
merging the position of the substance to be detected with an image representation of the examination object.

16. The method as claimed in claim 1, further comprising:
determining a quantitative variable for the substance to be detected.

17. A magnetic resonance apparatus, comprising:
a main magnet; and
a controller configured to detect a substance in an examination object via the magnetic resonance apparatus by:
receiving an instruction to detect the substance in the examination object;
determining a magnetic resonance sequence comprising a sub-sequence for detecting the substance as a function of the substance to be detected;
determining a measuring instant within the magnetic resonance sequence for capturing a respective magnetic resonance sequence signal to detect the substance as a function of the substance to be detected;
actuating the magnetic resonance apparatus to provide the determined magnetic resonance sequence;
actuating the magnetic resonance apparatus to capture the magnetic resonance sequence signal of the examination object at the measuring instant within the magnetic resonance sequence; and
detecting the substance when the magnetic resonance sequence signal fulfills a predetermined detection condition based upon an evaluation of the magnetic resonance sequence signal.

18. A non-transitory computer-readable medium having instructions stored thereon that, when executed by a controller of a magnetic resonance apparatus, cause the magnetic resonance apparatus to detect a substance in an examination object via the magnetic resonance apparatus by:
receiving an instruction to detect the substance in the examination object;
determining a magnetic resonance sequence comprising a sub-sequence for detecting the substance as a function of the substance to be detected;
determining a measuring instant within the magnetic resonance sequence for capturing a respective magnetic resonance sequence signal to detect the substance as a function of the substance to be detected;
actuating the magnetic resonance apparatus to provide the determined magnetic resonance sequence;
actuating the magnetic resonance apparatus to capture the magnetic resonance sequence signal of the examination object at the measuring instant within the magnetic resonance sequence; and
detecting the substance when the magnetic resonance sequence signal fulfills a predetermined detection condition based upon an evaluation of the magnetic resonance sequence signal.
